(19) United States
(12) Patent Application Publication
(10) Pub. No.: US 2004/0181088 A1
(43) Pub. Date: Sep. 16, 2004

(54) Production of lower aliphatic carboxylic acid ester

(76) Inventors: Kyoichi Watanabe, Oita-shi (JP); Hiroshi Uchida, Oita (JP)

Correspondence Address: SUGHRUE MION, PLLC, 2100 PENNSYLVANIA AVENUE, N.W., SUITE 800, WASHINGTON, DC, 20037, US

(21) Appl. No.: 10/415,947

(22) PCT Filed: Dec. 25, 2002
(86) PCT No.: PCT/JP02/13536

(30) Foreign Application Priority Data
Aug. 27, 2002 (JP) 2002-246062

(51) Int. Cl. C07C 67/04

(52) U.S. Cl. 560/241

(57) ABSTRACT

A process for producing a lower aliphatic carboxylic acid ester comprising reacting a lower aliphatic carboxylic acid and a lower olefin in the presence of a catalyst, wherein the raw materials contain substantially no halogens. The catalyst used can be remarkably prevented from deteriorating and a stable operation of the process can be continuously performed for an extended period.

### CROSS-REFERENCE TO RELATED APPLICATION

This application is an application filed under 35 U.S.C. §111(a) claiming benefit pursuant to 35 U.S.C. §119(e)(1) of the filing date of the Provisional Application 60/407,238 filed Sep. 3, 2002, pursuant to 35 §111(b).

### TECHNICAL FIELD

The present invention relates to a process for producing a lower aliphatic carboxylic acid ester by reacting a lower olefin and a lower aliphatic carboxylic acid and also relates to a lower aliphatic carboxylic acid ester obtained by the production process.

### BACKGROUND ART

As is well known, a corresponding lower aliphatic carboxylic acid ester can be obtained by reacting a lower olefin and a lower aliphatic carboxylic acid in the presence of an acid catalyst. It is also known that, in this reaction, a heteropolyacid and/or a heteropolyacid salt effectively acts as a catalyst. Specific examples of these conventional techniques include those described, for example, in Japanese Unexamined Patent Publications No. 4-139148 (JP-A-4-139148), No. 4-139149 (JP-A-4-139149), No. 5-65248 (JP-A-5-65248), No. 5-163200 (JP-A-5-163200), No. 5-170699 (JP-A-5-170699), No. 5-255185 (JP-A-5-255185), No. 5-294894 (JP-A-5-294894), No. 6-72951 (JP-A-6-72951) and No. 9-118647 (JP-A-9-118647). Thus, the development of catalysts having high initial activity is proceeding.

However, in the industrial production process, impurities derived from raw materials or by-products produced during the reaction give rise to deterioration of the catalyst and in turn cause problems such as reduction in the reaction result. Particularly, the catalyst deteriorates due to the effect of impurities contained in the raw materials with use of raw materials having a low purity or various impurities or by-products accumulated in the system after continuously performing a reaction for a long period of time by the process having a circulation system. This brings about, for example, a vicious circle of further accelerating the side reaction.

### DISCLOSURE OF INVENTION

The object of the present invention is to provide a process for producing a lower aliphatic carboxylic acid ester by esterifying a lower aliphatic carboxylic acid with a lower olefin in a gaseous phase, where the operation can be continuously and stably performed.

More specifically, the object of the present invention is to provide a process for producing a lower aliphatic carboxylic acid ester by esterifying a lower aliphatic carboxylic acid with a lower olefin in a gaseous phase, where the impurities derived from raw materials or the compounds derived from by-products produced in the process having a circulation system are reduced to a low concentration based on the raw materials to thereby prevent, particularly, the deterioration of a catalyst and enable a continuous and stable operation for a long period of time.

The present inventors have made extensive studies to find a process for producing a lower aliphatic carboxylic acid ester by reacting a lower olefin and a lower aliphatic carboxylic acid, where deterioration of the catalyst hardly occurs and the operation can be continuously and stably performed for a long period of time.

As a result, it has been found that, in the process for producing a lower aliphatic carboxylic acid ester by esterifying a lower aliphatic carboxylic acid and a lower olefin into a lower aliphatic carboxylic acid ester using a catalyst (B) in a gaseous phase, when the system is controlled to contain substantially no halogens, the catalyst can be remarkably prevented from deteriorating and in turn a stable operation can be continuously performed for a long period of time.

That is, the present invention (I) provides a process for producing a lower aliphatic carboxylic acid ester from a lower aliphatic carboxylic acid and a lower olefin in the presence of a catalyst (B), wherein the raw materials contain substantially no halogens.

The present invention (II) provides a lower aliphatic carboxylic acid ester produced by the production process of a lower aliphatic carboxylic acid ester of the present invention (I).

### BRIEF DESCRIPTION OF DRAWINGS

The each figure is a schematic view showing the process according to one embodiment for carrying out the present invention.

FIG. 1 is a view showing a one-path process having no circulation step.

FIG. 2 is a view showing a process having a circulation step from a post step.

### BEST MODE FOR CARRYING OUT THE INVENTION

The present invention is described in detail below. The present invention (I) is a process for producing a lower fatty carboxylic acid ester from a lower fatty carboxylic acid and a lower olefin in the presence of a catalyst (B), wherein the raw materials contain substantially no halogens.

The term “halogens” as used herein refers to elements belonging to Group 7B of the periodic table or compounds containing the elements. Specific examples thereof include fluorine, chlorine, bromine and iodine, and specific examples of the compounds containing these elements include hydrogen halides and alkyl halides. In particular, examples of the hydrogen halides include hydrogen chloride, and examples of the alkyl halides include methyl iodide.

Particularly when chlorine or iodine is present in the esterification reaction conditions for producing the lower aliphatic carboxylic acid ester, an extremely easily polymerizable substance may be disadvantageously produced as a by-product. The term “easily polymerizable substance” as used herein refers to an alkene having 4 or more carbon atoms or an oligomer. Of course, the substance is not limited thereto.

In the production process of a lower aliphatic carboxylic acid ester of the present invention, the raw materials are controlled to contain substantially no halogens and this is effective for reducing the deterioration rate of catalyst and, in turn, for continuously performing a stable operation for a long period of time.

The term “halogens in the raw materials” as used herein refers to halogens in the raw materials immediately before the inlet of a reactor in which the esterification reaction for producing a lower aliphatic carboxylic acid ester is performed.

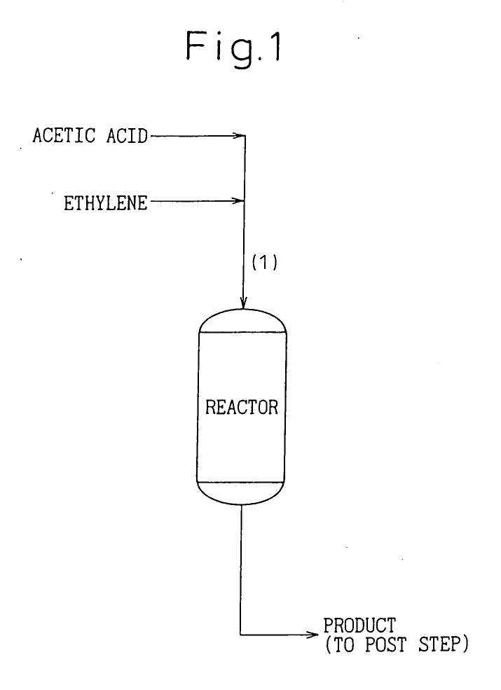

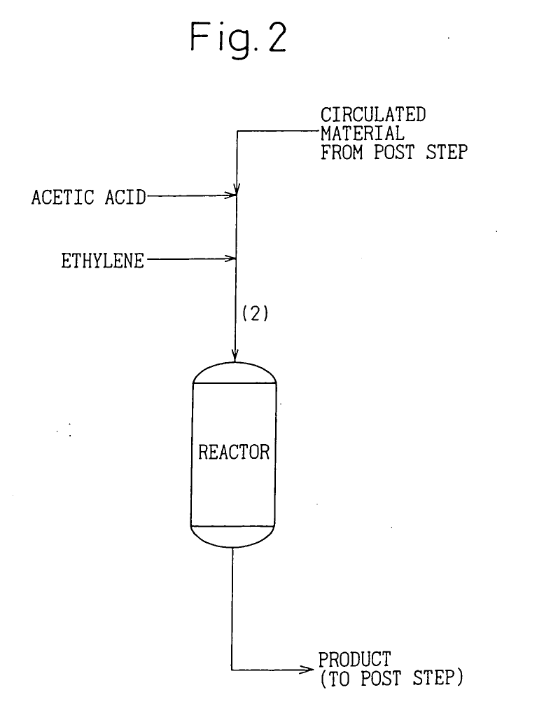

Specifically, for example, in the case where the reaction is performed by a one-path process having no circulation step, as shown in FIG. 1, the concentration immediately before the reactor inlet shown by (1) is indicated. In the case of a process having a circulation step from a post step as shown in FIG. 2, the concentration immediately before the reactor inlet shown by (2) is indicated. Of course, the present invention is not limited to these exemplified processes.

Accordingly, the term “raw materials” as used herein includes, in addition to newly fed lower olefin and acetic acid, unreacted gas after the reaction in the flow system, which is recovered through a post step and then fed to the reactor by the circulation system.

The position (1) in the process shown by FIG. 1 and the position (2) in the process shown by FIG. 2 are each generally kept at the same temperature as the reaction temperature in the reactor. Accordingly, in the measurement of concentration at such a position, the sampling, in particular, must be carefully designed. For example, the following method may be used. A part of a gas is sampled and cooled, the entire amount of the condensate collected is recovered and analyzed by gas chromatography, the effluent gas remaining uncondensed is measured on the flow rate of the gas flown out within the sampling time, and a part of the gas is sampled and analyzed on the composition by gas chromatography.

The term “substantially no” as used herein refers to a value of less than 1 ppm in the analysis of reaction gas described later.

In the present invention, the raw materials preferably contain substantially no halogens. In particular, if the concentration of halogens exceeds 20 ppm, the catalytic activity decreases at an extremely high rate and the catalyst life is largely shortened. The cause is not clearly known, however, it is considered that the presence of halogens incurs an increase in the production of easily polymerizable substance on the catalyst, this substance produces cokes, the cokes overwhelm the active sites of catalyst and, thereby, the catalyst is deactivated.

Accordingly, the concentration of halogens in the raw materials is preferably as low as possible and is preferably 20 ppm or less, more preferably 1 ppm or less. The method for controlling the concentration of halogens in the raw materials to 20 ppm or less is not particularly limited. Commonly known separation techniques may be used.

For example, fundamentally, the lower aliphatic carboxylic acids used as a raw material is of course refined to reduce the contents of these compounds as much as possible.

One representative example of the industrial production process of a lower aliphatic carboxylic acid is a method of reacting a lower alcohol and a carbon monoxide in the presence of a catalyst. Specific examples thereof include the method described in Japanese Examined Patent Publication No. 47-3334 (JP-B-47-3334). According to this method, an iodine compound is used as the activator of the catalyst for the production of a lower aliphatic carboxylic acid and therefore, halogens must be separated by performing thorough purification for obtaining a lower aliphatic carboxylic acid as a product. If a lower aliphatic carboxylic acid ester is produced using a lower aliphatic carboxylic acid obtained by such a method, the halogen concentration must be always controlled but the analysis take time and, depending on the case, re-purification becomes necessary and, as a result, the production cost increases.

On the other hand, the method dispensable with the analysis of halogens and capable of producing a lower aliphatic carboxylic acid ester with good efficiently is to use a lower aliphatic carboxylic acid produced by a method not using halogens. This can be realized by, for example, a process for producing an aliphatic carboxylic acid ester comprising the following first and second steps:

First Step

a step of reacting a lower olefin and oxygen in the presence of a catalyst (A) to obtain a lower aliphatic carboxylic acid containing no halogens;

Second Step

a step of reacting the lower aliphatic carboxylic acid containing no halogens obtained in the first step with a lower olefin in a gaseous phase in the presence of a catalyst (B) to obtain a lower aliphatic carboxylic acid ester.

The lower olefin used in the first and second steps is not particularly limited. A lower saturated hydrocarbon such as ethane and methane may be mixed therein. Preferably, a high-purity lower olefin is used.

The oxygen is also not particularly limited. An oxygen gas diluted with an inert gas such as nitrogen and carbon dioxide in, for example, the form of air may be fed. However, in the case of circulating the reaction gas, use of an oxygen having a high purity, preferably a purity of 99% or more, is generally advantageous.

The lower aliphatic carboxylic acid obtained in the first step is not particularly limited insofar as it is a lower aliphatic carboxylic acid, containing substantially no halogens, obtained by a reaction between a lower olefin and an oxygen in the presence of a catalyst (A).

The reaction form of a lower olefin and an oxygen is not particularly limited and a conventionally known reaction form can be selected. In general, an optimal method is selected depending on the catalyst used and the reaction is preferably performed by the method selected. For example, a liquid phase method may be selected for the reaction using an oxidation-reduction catalyst of a metal ion pair such as palladium-cobalt and iron disclosed in French Patent No. 1448361, and a gaseous phase method may be selected for the reaction using a catalyst containing palladium and at least one compound selected from a heteropolyacid and/or a salt thereof disclosed in Japanese Unexamined Patent Publications No. 7-89896 (JP-A-7-89896) and No. 9-67298 (JP-A-9-67298). Industrially, the gaseous phase method is preferred in view of the productivity.

The catalyst (A) for use in the first step is not particularly limited as long as it does not contain halogens, and a known catalyst may be used if it has an ability of reacting a lower olefin and oxygen to obtain a lower aliphatic carboxylic acid. Preferred is a catalyst comprising palladium and at least one compound selected from the group consisting of a heteropolyacid and a salt thereof.

The palladium may have any valence number but metal palladium is preferred. The term “metal palladium” as used herein refers to a palladium having a valence number of 0. The metal palladium can be usually obtained by reducing divalent and/or tetravalent palladium ions using a reducing agent such as hydrazine and hydrogen. At this time, there is no problem even if a some of the palladium is not in a metal state.

The raw material of the palladium is not particularly limited. Metal palladium can be of course used and a palladium compound capable of converting into metal palladium can also be used. Examples of the palladium compound capable of converting into metal palladium include halides (e.g., palladium chloride), organic acid salts (e.g., palladium acetate), palladium nitrate, palladium oxide, palladium sulfate and sodium tetrachloropalladate, however, the present invention is not limited thereto.

The lower aliphatic carboxylic acid of the present invention is an aliphatic carboxylic acid having from 1 to 4 carbon atoms and preferred examples thereof include formic acid, acetic acid, acrylic acid, propionic acid, methacrylic acid and a mixture of two or more thereof. Among these, acetic acid and acrylic acid are more preferred.

Examples of the lower olefin for use in the present invention include ethylene, propylene, n-butene, isobutene and a mixture of two or more thereof.

The catalyst (B) as used in the present invention is preferably a so-called acid catalyst. The term “acid catalyst” as used herein refers to a catalyst widely used in general, such as an ion-exchange resin, a mineral acid, a heteropolyacid, zeolite and a composite metal oxide. In particular, the catalyst (B) is suitably a heteropolyacid or a heteropolyacid salt.

The heteropolyacid used for the catalyst (A) and the catalyst (B) is a compound consisting of a center element and peripheral elements to which oxygen is bonded. The center element is usually silicon or phosphorus but may comprise any one atom selected from various atoms belonging to Groups 1 to 17 of the periodic table of elements. Specific examples thereof include a cupric ion; divalent beryllium, zinc, cobalt and nickel ions; trivalent boron, aluminum, gallium, iron, cerium, arsenic, antimony, phosphorus, bismuth, chromium and rhodium ions; tetravalent silicon, germanium, tin, titanium, zirconium, vanadium, sulfur, tellurium, manganese, nickel, platinum, thorium, hafnium and cerium ions and other rare earth ions; pentavalent phosphorus, arsenic, vanadium and antimony ions; hexavalent tellurium ion; and heptavalent iodide ion, however, the present invention is not limited thereto. Specific examples of the peripheral element include tungsten, molybdenum, vanadium, niobium and tantalum, however, the present invention is not limited thereto.

These heteropolyacids are known also as a “polyoxo-anion”, a “polyoxometallic salt” or a “metal oxide cluster”. Some structures of well-known anions are named after the researcher himself in this field, for example, Keggin, Wells-Dawson and Anderson-Evans-Perloff structure. These are described in detail in Poly-san no Kagaku, Kikan Kagaku Sosetsu (Chemistry of Polvacids, the Introduction of Chemistry quarterly), No. 20, compiled by Nippon Kagaku Kai (1993). The heteropolyacid usually has a high molecular weight, for example, a molecular weight of 700 to 8,500, and includes not only a monomer but also a dimeric complex.

The heteropolyacid salt is not particularly limited as long as it is a metal salt or onium salt resulting from substituting a part or all of the hydrogen atoms of the above-described heteropolyacid.

Specific examples thereof include metal salts such as lithium, sodium, potassium, cesium, magnesium, barium, copper, gold and gallium, and onium salts such as ammonia, however, the present invention is not limited thereto.

Particularly when the heteropolyacid is a free acid or a certain salt, the heteropolyacid exhibits a relatively high solubility in a polar solvent such as water or other oxygenated solvents. The solubility can be controlled by selecting an appropriate counter ion.

Preferred examples of the heteropolyacid which can be used as the catalyst in the present invention include:

| | |
|---|---|
| silicotungstic acid | H₄[SiW₁₂O₄₀].xH₂O |
| phosphotungstic acid | H₃[PW₁₂O₄₀].xH₂O |
| phosphomolybdic acid | H₃[PMo₁₂O₄₀].xH₂O |
| silicomolybdic acid | H₄[SiMo₁₂O₄₀].xH₂O |
| silicovanadotungstic acid | H₄₊ₙ[SiVₙW₁₂₋ₙO₄₀].xH₂O |
| phosphovanadotungstic acid | H₃₊ₙ[PVₙW₁₂₋ₙO₄₀].xH₂O |
| phosphovanadomolybdic acid | H₃₊ₙ[PVₙMo₁₂₋ₙO₄₀].xH₂O |
| silicovanadomolybdic acid | H₄₊ₙ[SiVₙMo₁₂₋ₙO₄₀].xH₂O |
| silicomolybdotungstic acid | H₄[SiMoₙW₁₂₋ₙO₄₀].xH₂O |
| phosphomolybdotungstic acid | H₃[PMoₙW₁₂₋ₙO₄₀].xH₂O |

wherein n is an integer of 1 to 11 and x is an integer of 1 or more, however, the present invention is not limited thereto.

Among these, preferred are silicotungstic acid, phosphotungstic acid, phosphomolybdic acid, silicomolybdic acid, silicovanadotungstic acid and phosphovanadotungstic acid, more preferred are silicotungstic acid, phosphotungstic acid, silicovanadotungstic acid and phosphovanadotungstic acid.

The method for synthesizing these heteropolyacids is not particularly limited and any method may be used. For example, the heteropolyacid can be obtained by heating an acidic aqueous solution (pH: approximately from 1 to 2) containing a salt of molybdic acid or tungstic acid and a simple oxygen acid of heteroatom or a salt thereof. For isolating the heteropolyacid compound from the resulting aqueous heteropolyacid solution, a method of crystallizing and separating the compound as a metal salt may be used. Specific examples thereof are described in Shin Jikken Kagaku Koza 8, Muki Kagobutsuno Gosei (III) (New Experimental Chemistry Course 8, Synthesis (III) of Inorganic Compounds), 3rd ed., compiled by Nippon Kagaku Kai, issued by Maruzen, page 1413 (Aug. 20, 1984), however, the present invention is not limited thereto. The Keggin structure of the heteropolyacid synthesized can be identified by the X-ray diffraction or the UV or IR measurement, in addition to the chemical analysis.

Particularly preferred examples of the heteropolyacid salt include a lithium salt, a sodium salt, a potassium salt, a cesium salt, a magnesium salt, a barium salt, a copper salt, a gold salt, a gallium salt and an ammonium salt of the above-described preferred heteropolyacids. Among these, more preferred are a lithium salt of silicotungstic acid and a cesium salt of phosphotungstic acid.

Specific examples of the heteropolyacid salt include a lithium salt of silicotungstic acid, a sodium salt of silicotungstic acid, a copper salt of silicotungstic acid, a gold salt of silicotungstic acid, a gallium salt of silicotungstic acid, a lithium salt of phosphotungstic acid, a sodium salt of phosphotungstic acid, a copper salt of phosphotungstic acid, a gold salt of phosphotungstic acid, a gallium salt of phosphotungstic acid, a lithium salt of phosphomolybdic acid, a sodium salt of phosphomolybdic acid, a copper salt of phosphomolybdic acid, a gold salt of phosphomolybdic acid, a gallium salt of phosphomolybdic acid, a lithium salt of silicomolybdic acid, a sodium salt of silicomolybdic acid, a copper salt of silicomolybdic acid, a gold salt of silicomolybdic acid, a gallium salt of silicomolybdic acid, a lithium salt of silicovanadotungstic acid, a sodium salt of silicovanadotungstic acid, a copper salt of silicovanadotungstic acid, a gold salt of silicovanadotungstic acid, a gallium salt of silicovanadotungstic acid, a lithium salt of phosphovanadotungstic acid, a sodium salt of phosphovanadotungstic acid, a copper salt of phosphovanadotungstic acid, a gold salt of phosphovanadotungstic acid, a gallium salt of phosphovanadotungstic acid, a lithium salt of phosphovanadomolybdic acid, a sodium salt of phosphovanadomolybdic acid, a copper salt of phosphovanadomolybdic acid, a gold salt of phosphovanadomolybdic acid, a gallium salt of phosphovanadomolybdic acid, a lithium salt of silicovanadomolybdic acid, a sodium salt of silicovanadomolybdic acid, a copper salt of silicovanadomolybdic acid, a gold salt of silicovanadomolybdic acid and a gallium salt of silicovanadomolybdic acid.

Among these, preferred are a lithium salt of silicotungstic acid, a sodium salt of silicotungstic acid, a copper salt of silicotungstic acid, a gold salt of silicotungstic acid, a gallium salt of silicotungstic acid, a lithium salt of phosphotungstic acid, a sodium salt of phosphotungstic acid, a copper salt of phosphotungstic acid, a gold salt of phosphotungstic acid, a gallium salt of phosphotungstic acid, a lithium salt of phosphomolybdic acid, a sodium salt of phosphomolybdic acid, a copper salt of phosphomolybdic acid, a gold salt of phosphomolybdic acid, a gallium salt of phosphomolybdic acid, a lithium salt of silicomolybdic acid, a sodium salt of silicomolybdic acid, a copper salt of silicomolybdic acid, a gold salt of silicomolybdic acid, a gallium salt of silicomolybdic acid, a lithium salt of silicovanadotungstic acid, a sodium salt of silicovanadotungstic acid, a copper salt of silicovanadotungstic acid, a gold salt of silicovanadotungstic acid, a gallium salt of silicovanadotungstic acid, a lithium salt of phosphovanadotungstic acid, a sodium salt of phosphovanadotungstic acid, a copper salt of phosphovanadotungstic acid, a gold salt of phosphovanadotunstic acid and a gallium salt of phosphovanadotungstic acid.

More preferred are a lithium salt of silicotungstic acid, a sodium salt of silicotungstic acid, a copper salt of silicotungstic acid, a gold salt of silicotungstic acid, a gallium salt of silicotungstic acid, a lithium salt of phosphotungstic acid, a sodium salt of phosphotungstic acid, a copper salt of phosphotungstic acid, a gold salt of phosphotungstic acid, a gallium salt of phosphotungstic acid, a lithium salt of silicovanadotungstic acid, a sodium salt of silicovanadotungstic acid, a copper salt of silicovanadotungstic acid, a gold salt of silicovanadotungstic acid, a gallium salt of silicovanadotungstic acid, a lithium salt of phosphovanadotungstic acid, a sodium salt of phosphovanadotungstic acid, a copper salt of phosphovanadotungstic acid, a gold salt of phosphovanadotungstic acid and a gallium salt of phosphovanadotungstic acid.

The catalyst (A) may be used after loading it on a support. The method for loading is not particularly limited and any method may be used. For example, in the case of loading a palladium compound capable of converting into a metal palladium, a method of dissolving the palladium compound in an appropriate solvent such as water or acetone or in an inorganic or organic acid such as hydrochloric acid, nitric acid or acetic acid or a solution thereof, impregnating the solution into the support and drying the support may be used.

The reaction temperature at the production of a lower aliphatic carboxylic acid in the first step is not particularly limited. The reaction temperature is preferably from 100 to 300° C., more preferably from 120 to 250° C. In view of equipment, the reaction pressure in practice is advantageously from 0.0 MPa (gauge pressure) to 3.0 MPa (gauge pressure), however, the reaction pressure is not particularly limited. The reaction pressure is more preferably from 0.1 MPa (gauge pressure) to 1.5 MPa (gauge pressure).

The reaction raw material gas for use in the first step contains a lower olefin and oxygen and if desired, nitrogen, carbon dioxide or a rare gas can be used as a diluent. To a reactor for producing a lower aliphatic carboxylic acid, the lower olefin is fed in an amount of giving a proportion of 5 to 80 vol %, preferably from 8 to 50 vol %, and the oxygen is fed in an amount of giving a proportion of 1 to 15 vol %, preferably from 3 to 12 vol %, based on the reaction raw material gas. Depending on the catalyst, the presence of water in the reaction gas provides an effect to elevate the activity of producing a lower aliphatic carboxylic acid and maintain the catalytic activity. In this case, the water is suitably contained in the reaction gas in the range from 1 to 50 vol %, preferably from 5 to 40 vol %.

In the standard state, the reaction raw material gas is preferably passed through the catalyst (A) at a space velocity of 10 to 15,000 hr⁻¹, more preferably from 300 to 8,000 hr⁻¹.

The method for obtaining a lower aliphatic carboxylic acid, particularly acetic acid, using the catalyst (A) is described in detail in Japanese Unexamined Patent Publications No. 7-89896 (JP-A-7-89896), No. 9-67298 (JP-A-9-67298) and No. 11-347412 (JP-A-11-347412).

The catalyst (B) may be also used after loading it on a support. In this case, the catalyst (B) content is preferably from 10 to 200 mass %, more preferably from 50 to 150 mass %, based on the entire mass of the support.

If the catalyst (B) content is less than 10 mass %, the content of active components in the catalyst may be excessively small and the activity per catalyst unit mass may disadvantageously decrease.

If the catalyst (B) content exceeds 200 mass %, the effective surface area may decrease, as a result, the effect obtainable by the increase in the supported amount may not be brought out and at the same time, coking may be readily generated to greatly shorten the catalyst life.

The catalyst (B) for use in the present invention can be produced by a desired method. For example, the method for producing a heteropolyacid and/or heteropolyacid salt catalyst is described below.

Step (A):

This is a step for obtaining a solution or suspension of a heteropolyacid and/or heteropolyacid salt.

Step (B):

This is a step for loading the solution or suspension obtained in the step (A) on a support.

The solvent which can be used in the step (A) is not particularly limited as long as it can uniformly dissolve or suspend the desired heteropolyacid and/or heteropolyacid salt, and for example, water, an organic solvent or a mixture thereof may be used. Preferred examples of the solvent include water, alcohols and lower aliphatic carboxylic acids, however, the present invention is not limited thereto.

The method for dissolving or suspending a heteropolyacid and/or a heteropolyacid salt in the solvent is not particularly limited and any method may be used as long as it can uniformly dissolve or suspend the desired heteropolyacid and/or heteropolyacid salt.

The optimal volume of the solution or suspension varies depending on the loading method in the step (B) and the support used but this is not particularly limited.

The step (B) is a step for loading a solution or suspension of a heteropolyacid and/or a heteropolyacid salt obtained in the step (A) on a support to obtain a catalyst for use in the production of a lower aliphatic carboxylic acid ester.

The method for loading the solution or suspension of a heteropolyacid and/or a heteropolyacid salt on a support is not particularly limited and a known method may be used.

For example, the catalyst may be prepared by dissolving or suspending a heteropolyacid and/or a heteropolyacid salt in a solvent to obtain a solution or suspension corresponding to the liquid absorption amount of a support and impregnating the solution or suspension into the support.

The catalyst may also be prepared by using an excess solution or suspension, impregnating it into a support while appropriately moving the support in the heteropolyacid solution and then removing the excess acid through filtration.

In the case of loading a heteropolyacid salt, a method of loading a heteropolyacid and at the same time, forming it into a salt using an element contained in the support and capable of forming a salt may also be used, in addition to the above-described method of previously preparing a heteropolyacid salt and then loading it.

The thus-obtained wet catalyst is preferably dried by placing it in a heating oven for a few hours. Thereafter, the catalyst is cooled to the ambient temperature in a desiccator. If the drying temperature exceeds about 400° C., the skeleton of the heteropolyacid is disadvantageously destroyed. The drying temperature is preferably from 80 to 350° C.

Industrially, the catalyst may be continuously dried using a dryer such as through-flow rotary dryer, continuous fluidized bed dryer or continuous hot air carrier type dryer.

The amount of the heteropolyacid supported can be calculated simply by subtracting the mass of the support used from the dry mass of the catalyst prepared. A more exact amount can be measured by chemical analysis such as ICP (induction coupled plasma emission spectrometry).

In practicing the production process of a lower aliphatic carboxylic acid ester of the present invention, the ratio between the lower olefin and the lower aliphatic carboxylic acid used is preferably such that the lower olefin is in an equimolar amount or excess molar amount to the lower aliphatic carboxylic acid. The ratio of lower olefin:lower aliphatic carboxylic acid is preferably, as a molar ratio, from 1:1 to 30:1, more preferably from 3:1 to 20:1, still more preferably from 5:1 to 15:1.

In the production process of a lower aliphatic carboxylic acid ester of the present invention, the gaseous phase reaction may be performed in either a fixed bed system or a fluidized bed system. The shape of the support may also be selected from those formed into a size from powder to a few mm according to the reaction system employed in practice.

In the production process of a lower aliphatic carboxylic acid ester of the present invention, it is preferred in view of catalyst life to mix a slight amount of water in the raw materials. However, if an excessively large amount of water is added, by-products such as an alcohol and an ether disadvantageously increase. In general, the amount of water is preferably from 1 to 15 mol %, more preferably from 2 to 8 mol %, based on the entire amount of the olefin and lower aliphatic carboxylic acid used.

The reaction temperature and the reaction pressure must be in the range of keeping the gaseous form of the feed medium and vary depending on the raw materials used. In general, the reaction temperature is preferably from 120 to 250° C., more preferably from 140 to 220° C.

The pressure is preferably from atmospheric pressure to 3 MPa (gauge pressure), more preferably from atmospheric pressure to 2 MPa (gauge pressure).

With respect to the space velocity (hereinafter simply referred to as “GHSV”) of the raw materials fed to the catalyst, the raw materials are preferably passed through the catalyst layer at a GHSV of 100 to 7,000 hr⁻¹, more preferably from 300 to 3,000 hr⁻¹.

The shape of the substance which can be used as the support for the catalyst of the present invention is not particularly limited, however, those capable of providing, when prepared as a catalyst after loading the catalyst component, a catalyst having a specific surface area by the BET method of 65 to 350 m²/g are preferred. Specifically, powder, spheres, pellets and other arbitrary forms may be used. Examples of the substance include silica, kieselguhr, montmorillonite, titania, activated carbon, alumina and silica alumina, however, the present invention is not limited thereto.

The support is preferably a support comprising a siliceous main component and having a spherical or pellet form. The support is preferably a silica having a purity of 85 mass % or more, more preferably 95 mass % or more, based on the entire mass of the support and at the same time, having a compression strength of 30 N or more. The “compression strength” as used herein can be measured in accordance with, for example, JIS Z 8841 “Granulated Material—Strength Test Method”.

The average diameter thereof varies depending on the reaction form but is preferably from 2 to 10 mm in the case of a fixed bed and from powder to 5 mm in the case of a fluid bed.

The present invention is further illustrated below by referring to Examples and Reference Example, however, these Examples are only for describing the outline of the present invention and the present invention should not be construed as being limited thereto.

Analysis of Reaction Gas

The concentration of halogens in the gas fed to a reaction tube was analyzed, using gas chromatograph GC-14B with an electron capture-type detector manufactured by Shimadzu Corporation, by sampling a part of feed gas under heating not to cause condensation and directly introducing it to a 1-ml gas sampler (MGS-4).

The gas at the outlet of the reaction tube was analyzed as follows. The whole amount of the gas was cooled, the whole amount of the condensed reaction solution collected was recovered, 1 ml of 1,4-dioxane as the internal standard was added to 10 ml of the reaction solution to prepare an analysis solution, and 0.2 μl of the analysis solution was injected and analyzed using gas chromatograph GC-14B manufactured by Shimadzu Corporation.

As for the effluent gas remaining uncondensed, the flow rate of the outlet gas flown out within the sampling time was measured, 50 ml of the gas was sampled, the whole amount was passed to a 1-ml gas sampler (MGS-4) attached to gas chromatograph GC-14B manufactured by Shimadzu Corporation, and the composition was analyzed by gas chromatography.

### Preparation of Catalyst (a) as One Example of Catalyst (A)

<Support>

Silica (KA-1, produced by Sud-chemie) was used.

<Preparation Method>

2.81 g of sodium tetrachloropalladate, 1.05 g of chloroauric acid and 0.1402 g of zinc chloride were weighed and thereto, pure water was added and dissolved to obtain 45 ml of Aqueous Solution (1). To Aqueous Solution (1), 100 ml of the support was added and impregnated with the solution by thoroughly stirring.

Separately, 8.00 g of sodium metasilicate was weighed and thereto, 100 g of pure water was added and dissolved to prepare Aqueous Solution (2). To Aqueous Solution (2), the support impregnated with Aqueous Solution (1) was added, left standing at room temperature for 20 hours and thereto, 8.00 g of hydrazine monohydrate was gradually added at room temperature while stirring. The resulting solution was stirred for 4 hours. Thereafter, the catalyst was collected by filtration, washed by passing pure water for 40 hours and then dried at 110° C. for 4 hours in an air stream.

Subsequently, 0.266 g of sodium tellurite was weighed and thereto, 45 g of pure water was added to prepare Aqueous Solution (3). To Aqueous Solution (3), the metal palladium-supported catalyst prepared above was added and impregnated with the whole amount of Aqueous Solution (3). Thereafter, the catalyst was dried at 110° C. for 4 hours in an air stream to obtain a tellurium-added metal palladium-supported catalyst.

Separately, 23.98 g of silicotungstic acid was weighed and thereto, pure water was added and dissolved to make 45 ml, thereby preparing Aqueous Solution (4). Thereto, the tellurium-added metal palladium-supported catalyst prepared above was added, impregnated with the whole amount of Aqueous Solution (4) and then dried at 110° C. for 4 hours in an air stream to obtain Catalyst (a).

### Preparation of Catalyst (b) as One Example of Catalyst (B)

<Support>

Synthetic silica (CARiACT Q-10, produced by Fuji Silysia Chemical Ltd.) (specific surface area: 219.8 m²/g, pore volume: 0.660 cm³/g) was used.

<Preparation Method>

The support was dried for 4 hours in a (hot air) dryer adjusted to 110° C. 34.99 g of silicotungstic acid and 0.0837 g of lithium nitrate were weighed, 15 ml of pure water was added thereto, and the mixture was uniformly dissolved to obtain an aqueous Li_{0.1}H_{2.9}PW₁₂O₄₀ solution (impregnating solution). To the impregnating solution, 100 ml of the support was added and thoroughly stirred. The support impregnated with the solution was air dried for 1 hour and thereafter dried for 5 hours by a dryer adjusted to 150° C. to obtain Catalyst (b).

### EXAMPLE 1

After filling 40 ml of Catalyst (b) in a reaction tube, 58.98 g/hr of high-purity ethylene, 12.87 g/hr of a commercially available high-purity acetic acid, 2.17 g/hr of pure water and 6.75 g/hr of nitrogen, each in a gaseous form, were passed through the reaction tube at a pressure of 0.8 MPa (gauge pressure) and the reaction was performed while keeping the highest temperature portion of the catalyst layer at 165° C. At this time, halogens in the gas at the inlet of reaction tube were measured by a gas chromatograph with an electron capture-type detector and it was confirmed that halogens were not detected. After cooling the gas at the outlet of reactor, uncondensed unreacted ethylene and the like were separated by a gas-liquid separator to obtain a reaction product containing ethyl acetate. The results are shown in Table 1 below.

TABLE 1

| | Concentration of Halogens in | Reaction | STY of Ethyl | Activity Reduction | |
|---|---|---|---|---|---|
| | Gas at Inlet of Reaction Tube | Time | Acetate | Rate | Amount of Butene |
| | (ppm) | (hr) | (g/L-hr) | (STY drop/100 hr) | Produced (%) |
| Example 1 | 0 | 5 | 243 | 0.3 | 0.01138 |
| | | 403 | 241.8 | | |
| Example 2 | 0 | 5 | 244.8 | 0.28 | 0.01053 |
| | | 382 | 243.7 | | |
| Comparative | 1020 (hydrogen chloride) | 5 | 243.2 | 1.81 | 0.30154 |
| Example 1 | | 410 | 235.9 | | |
| Comparative | 22 (hydrogen chloride) | 5 | 239.6 | 0.53 | 0.03534 |
| Example 2 | | 395 | 237.5 | | |
| Comparative | 16 (methyl iodide) | 5 | 241.1 | 0.41 | 0.02416 |
| Example 3 | | 405 | 239.5 | | |

The amount of butene produced is shown by the concentration of butene in gas at the outlet of reaction tube.

The butene concentration is a total value of 1-butene, cis-2-butene and trans-2-butene.

### EXAMPLE 2

A reaction apparatus capable of recycling unreacted gas was used and 40 ml of Catalyst (a) was filled in the reaction tube. Thereafter, 1,408 g/hr of high-purity ethylene and recycled ethylene, 616 g/hr of oxygen, 5,338 g/hr of pure water, 6,256 g/hr of nitrogen and 1,097 g/hr of recycled carbon dioxide gas were passed through the reaction tube at a pressure of 0.8 MPa (gauge pressure) and the reaction was performed while keeping the highest temperature portion of the catalyst layer at 200° C.

After cooling the gas at the outlet of reactor, uncondensed unreacted ethylene and carbon dioxide gas as a by-product were separated by a gas-liquid separator to obtain a condensed product. Halogens in the condensed product were measured by a gas chromatograph with an electron capture-type detector but were not detected. This condensed product was purified by distillation to obtain an acetic acid having a purity of 99.99%. A part of the uncondensed gas was discarded so as to prevent accumulation of carbon dioxide gas and the remaining gas was recycled to the reaction tube.

Thereafter, the reaction was performed in the same manner as in Example 1 except that the acetic acid containing no halogens obtained above was used in place of the commercially available high-purity acetic acid of Example 1. Then, a reaction product containing ethyl acetate was obtained. The results are shown in Table 1.

The results obtained are the same as those in Example 1.

### COMPARATIVE EXAMPLE 1

The same reaction as in Example 1 was performed except for adding a slight amount of hydrogen chloride to the commercially available high-purity acetic acid. The concentration of hydrogen chloride in the gas at the inlet of reaction tube was 1,020 ppm. The results are shown in Table 1.

As compared with Examples 1 and 2, the amount of by-product is large and the activity reduction rate is high.

### COMPARATIVE EXAMPLE 2

The same reaction as in Example 1 was performed except for adding a slight amount of hydrogen chloride to the commercially available high-purity acetic acid. The concentration of hydrogen chloride in the gas at the inlet of reaction tube was 22 ppm. The results are shown in Table 1.

As compared with Examples 1 and 2, the amount of by-product is slightly large but the activity reduction rate is almost the same.

### COMPARATIVE EXAMPLE 3

The same reaction as in Example 1 was performed except for adding a slight amount of methyl iodide to the commercially available high-purity acetic acid. The concentration of methyl iodide in the gas at the inlet of reaction tube was 16 ppm. The results are shown in Table 1.

As compared with Examples 1 and 2, the amount of by-product is slightly large but the activity reduction rate is almost the same.

As is apparent from the results above, in the process for producing a lower aliphatic carboxylic acid ester from a lower aliphatic carboxylic acid and a lower olefin in the presence of an acid catalyst, a stable operation can be continuously performed, for a long period of time, by controlling the concentration of halogens in the raw materials to 20 ppm or less. Furthermore, use of a lower aliphatic carboxylic acid produced by a process using substantially no halogens eliminates risks and is effective for continuously performing a stable operation.

1. A process for producing a lower aliphatic carboxylic acid ester from a lower aliphatic carboxylic acid and a lower olefin in the presence of a catalyst (B), wherein the raw materials contain substantially no halogens.

2. The process as claimed in claim 1, wherein the concentration of halogens is 20 ppm or less.

3. The process as claimed in claim 1, wherein the concentration of halogens is 1 ppm or less.

4. The process as claimed in any one of claims 1 to 3, wherein the halogen is at least one compound of an element selected from the group consisting of fluorine, chlorine, bromine and iodine.

5. The process as claimed in any one of claims 1 to 3, wherein the halogen is a hydrogen halide.

6. The process as claimed in claim 5, wherein the hydrogen halide is hydrogen chloride.

7. The process as claimed in any one of claims 1 to 3, wherein the halogen is an alkyl halide.

8. The process as claimed in claim 7, wherein the alkyl halide is methyl iodide.

9. The process as claimed in any one of claims 1 to 3, wherein the halogen is originated in water or a lower aliphatic carboxylic acid which are constituent components of the reaction raw materials.

10. The process as claimed in any one of claims 1 to 9, which comprises the following first and second steps:
First Step
a step of reacting a lower olefin and oxygen in the presence of a catalyst (A) to obtain a lower aliphatic carboxylic acid containing no halogens;
Second Step
a step of reacting the lower aliphatic carboxylic acid containing no halogens obtained in the first step with a lower olefin in a gaseous phase in the presence of a catalyst (B) to obtain a lower aliphatic carboxylic acid ester.

11. The process as claimed in any one of claims 1 to 10, wherein the lower aliphatic carboxylic acid is at least one member selected from the group consisting of lower aliphatic carboxylic acids having from 1 to 4 carbon atoms and a mixture of two or more thereof.

12. The process as claimed in any one of claims 1 to 11, wherein the lower olefin is at least one member selected from the group consisting of ethylene, propylene, n-butene, isobutene and a mixture of two or more thereof.

13. The process as claimed in any one of claims 1 to 12, wherein the catalyst (A) comprises palladium and at least one compound selected from heteropolyacid and heteropolyacid salts.

14. The process for producing a lower fatty carboxylic acid ester as claimed in any one of claims 1 to 13, wherein the catalyst (B) comprises at least one compound selected from a heteropolyacid and/or a heteropolyacid salt.

15. The process as claimed in claim 13 or 14, wherein the heteropolyacid contains at least one compound selected from the group consisting of silicotungstic acid, phosphotungstic acid, phosphomolybdic acid, silicomolybdic acid, silicovanadotungstic acid, phosphovanadotungstic acid and phosphovanadomolybdic acid.

16. The process as claimed in claim 13 or 14, wherein the heteropolyacid salt contains at least one compound selected from the group consisting of a lithium salt, a sodium salt, a potassium salt, a cesium salt, a magnesium salt, a barium salt, a copper salt, a gold salt, a gallium salt and an ammonium salt of silicotungstic acid, phosphotungstic acid, phosphomolybdic acid, silicomolybdic acid, silicovanadotungstic acid, phosphovanadotungstic acid and phosphovanadomolybdic acid.

17. The process as claimed in any one of claims 1 to 16, wherein the reaction between the lower olefin and the lower aliphatic carboxylic acid is performed in the presence of water rendered not to contain halogens.

18. A lower aliphatic carboxylic acid ester produced by a production process as set forth in any one of claims 1 to 17.
